(19) United States
(12) Patent Application Publication
(10) Pub. No.: US 2018/0370461 A1
(43) Pub. Date: Dec. 27, 2018

(54) VEHICLE SYSTEM FOR REDUCING MOTION SICKNESS

(71) Applicant: MAGNA ELECTRONICS INC., Auburn Hills, MI (US)

(72) Inventors: Martin Solar, Erlenbach (DE); Michael Junglas, Elsenfeld (DE)

(21) Appl. No.: 16/014,580

(22) Filed: Jun. 21, 2018

Related U.S. Application Data
(60) Provisional application No. 62/523,963, filed on Jun. 23, 2017.

(51) Int. Cl. B60R 16/023

(52) U.S. Cl. CPC B60R 16/0232; A61M 2021/005

(57) ABSTRACT

A vehicular control system includes a plurality of sensors disposed in a vehicle and having respective fields of sensing that encompass occupants in the vehicle. A control includes a processor operable to process data captured by the sensors. Responsive to processing of data captured by the sensors, the control determines a likelihood that an individual occupant is developing motion sickness. Responsive to determination that an individual occupant is developing motion sickness, the control at least one of (i) determines an optimized driving route to reduce the causes of motion sickness, (ii) generates a message to the determined individual occupant developing motion sickness, and (iii) generates a display of images for viewing by the determined individual occupant developing motion sickness.

### CROSS REFERENCE TO RELATED APPLICATION

The present application claims the filing benefits of U.S. provisional application Ser. No. 62/523,963, filed Jun. 23, 2017, which is hereby incorporated herein by reference in its entirety.

### FIELD OF THE INVENTION

The present invention relates generally to a vehicle driving assistance system for a vehicle and, more particularly, to a vehicle driving assistance system that utilizes one or more cameras at a vehicle.

### BACKGROUND OF THE INVENTION

Use of sensors in vehicle systems is common and known, such as for monitoring a driver of a vehicle or monitoring a cabin or interior space of a vehicle. Examples of such known systems are described in U.S. Pat. Nos. 8,258,932; 6,166,625 and/or 6,485,081, which are hereby incorporated herein by reference in their entireties.

### SUMMARY OF THE INVENTION

The present invention provides a driving assistance system or control system for a vehicle that utilizes one or more cameras or other sensors to capture data representative of passengers in a cabin of the vehicle, and provides a control that processes data captured by the sensor(s) to determine a likelihood that an individual non-driving passenger is getting or developing motion sickness. Responsive to determination that an individual non-driving passenger is getting or developing motion sickness, the control at least one of (i) determines an optimized driving route to reduce the causes of motion sickness, (ii) generates a message to the determined individual non-driving passenger developing motion sickness, and (iii) generates a display of images for viewing by the determined individual non-driving passenger developing motion sickness, with the displayed images providing a display of the exterior scene in real-time. Thus, the system provides or generates outputs that help to reduce the motion sickness of one or more passengers of the vehicle.

These and other objects, advantages, purposes and features of the present invention will become apparent upon review of the following specification in conjunction with the drawings.

### BRIEF DESCRIPTION OF THE DRAWINGS

FIG. 1 is a plan view of a vehicle with a vision system that incorporates cameras in accordance with the present invention; and

FIG. 2 shows an autonomous concept vehicle cabin with open side doors with four opposing passenger seats and video screens integrated into the interior, including the door panel's inside.

### DESCRIPTION OF THE PREFERRED EMBODIMENTS

A vehicle vision system and/or driver assist system and/or object detection system and/or alert system operates to capture images exterior of the vehicle and may process the captured image data to display images and to detect objects at or near the vehicle and in the predicted path of the vehicle, such as to assist a driver of the vehicle in maneuvering the vehicle in a rearward direction. The vision system includes an image processor or image processing system that is operable to receive image data from one or more cameras and provide an output to a display device for displaying images representative of the captured image data. Optionally, the vision system may provide display, such as a rearview display or a top down or bird's eye or surround view display or the like.

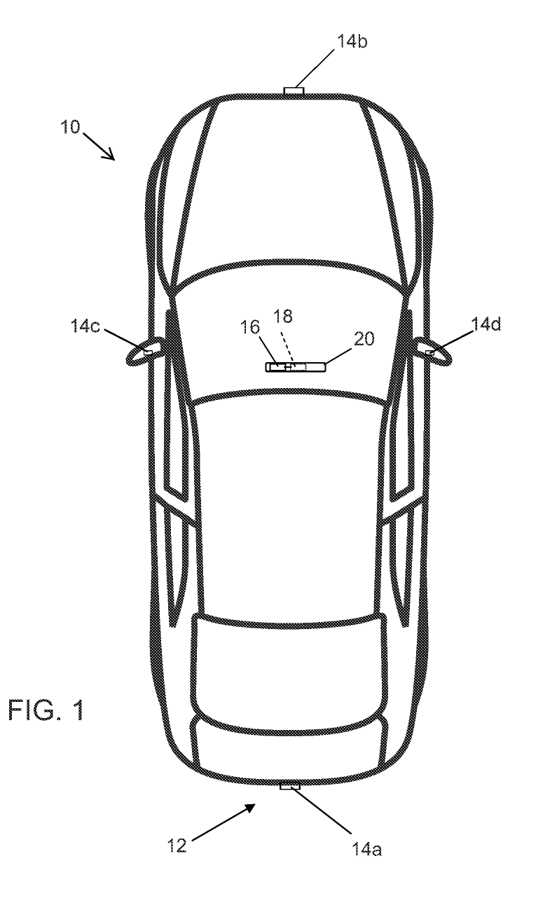

Referring now to the drawings and the illustrative embodiments depicted therein, a vehicle 10 includes an imaging system or vision system 12 that includes at least one exterior viewing imaging sensor or camera, such as a rearward viewing imaging sensor or camera 14a (and the system may optionally include multiple exterior viewing imaging sensors or cameras, such as a forward viewing camera 14b at the front (or at the windshield) of the vehicle, and a sideward/rearward viewing camera 14c, 14d at respective sides of the vehicle), which captures images exterior of the vehicle, with the camera having a lens for focusing images at or onto an imaging array or imaging plane or imager of the camera (FIG. 1). Optionally, a forward viewing camera may be disposed at the windshield of the vehicle and view through the windshield and forward of the vehicle, such as for a machine vision system (such as for traffic sign recognition, headlamp control, pedestrian detection, collision avoidance, lane marker detection and/or the like). The vision system 12 includes a control or electronic control unit (ECU) or processor 18 that is operable to process image data captured by the camera or cameras and may detect objects or the like and/or provide displayed images at a display device 16 for viewing by the driver of the vehicle (although shown in FIG. 1 as being part of or incorporated in or at an interior rearview mirror assembly 20 of the vehicle, the control and/or the display device may be disposed elsewhere at or in the vehicle). The data transfer or signal communication from the camera to the ECU may comprise any suitable data or communication link, such as a vehicle network bus or the like of the equipped vehicle.

For autonomous vehicles suitable for deployment with the system of the present invention, an occupant of the vehicle may, under particular circumstances, be desired or required to take over operation/control of the vehicle and drive the vehicle so as to avoid potential hazard for as long as the autonomous system relinquishes such control or driving. Such occupant of the vehicle thus becomes the driver of the autonomous vehicle. As used herein, the term “driver” refers to such an occupant, even when that occupant is not actually driving the vehicle, but is situated in the vehicle so as to be able to take over control and function as the driver of the vehicle when the vehicle control system hands over control to the occupant or driver or when the vehicle control system is not operating in an autonomous or semi-autonomous mode.

Typically an autonomous vehicle would be equipped with a suite of sensors, including multiple machine vision cameras deployed at the front, sides and rear of the vehicle, multiple radar sensors deployed at the front, sides and rear of the vehicle, and/or multiple lidar sensors deployed at the front, sides and rear of the vehicle. Typically, such an autonomous vehicle will also have wireless two way communication with other vehicles or infrastructure, such as via a car2car (V2V) or car2x communication system.

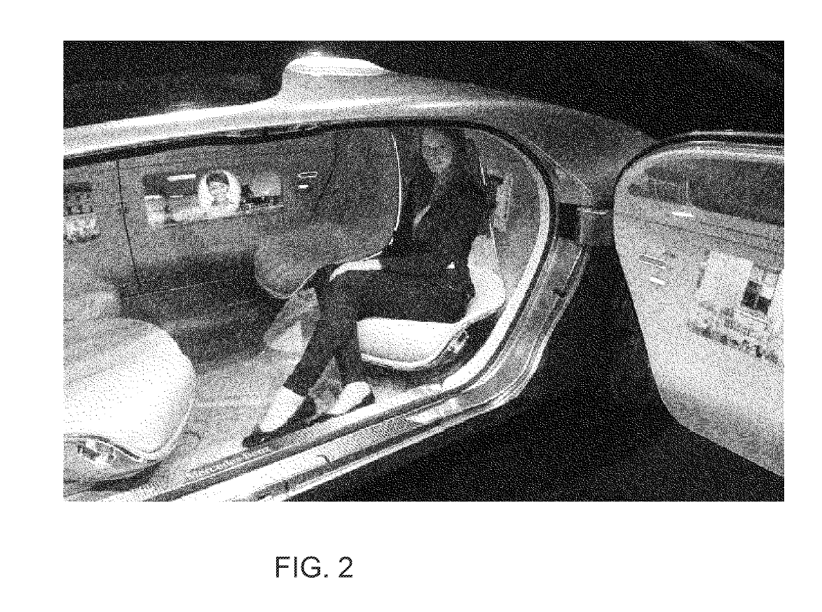

Newly presented autonomous vehicle concepts show uncommon passenger compartment layouts and seat arrangements. The compartments turn more and more into offices or living rooms on wheels. In some concepts, not all seats are turned into the frontal driving direction, but some are turned with the backrest to the front, such as shown in FIG. 2. While the driver is released from the driving task, the majority of the passengers are concentrating on things going on the inside of the compartment, rather than looking out of the windows. Due to that, the causes of the decelerations and accelerations sensed in the occupant's vestibular system are no longer matching to what the occupant's visual system is conceiving. Thus, some passengers may tend to develop motion sickness when staring at in-cabin computer screens.

For preventing passengers of autonomously driven vehicles to become motion sick due to being detached from the motion visible at the outside of the vehicle, the autonomous vehicle system according the present invention may adapt the driving style of the vehicle in a manner to minimize the causes of motion sickness, which are essentially the accelerations and decelerations. Optionally, the system may also adapt and optimize the higher harmonics of accelerations and decelerations, both longitudinal and lateral. The system may also select a route or path to the desired driving destination that leads to terrain that has the least amount of curves or the least degree of curves, that is the least hilly, and/or that has the smoothest underground or road surface (e.g., selecting a paved road instead of a dirt or gravel road) with an optimization algorithm. Optionally, the optimization algorithm may be any kind of network or artificial intelligence (AI) or an optimal control algorithm using the objectives as weighted optimization objectives.

The system according the invention may also collect all passengers' body and health data via a vehicle occupant monitoring and data processing system, including data pertaining to the pupils opening, the skin resistance, the (cold) transpiration, the body temperature, and the heart beat rate.

The monitoring system may utilize aspects of the monitoring systems described in U.S. Pat. Nos. 8,258,932; 6,166,625 and/or 6,485,081, and/or U.S. Publication Nos. US-2017-0274906; US-2015-0296135 and/or US-2015-0294169, which are hereby incorporated herein by reference in their entireties. The occupant monitoring system may include various sensors in the vehicle, such as microphone sensors and image sensors and infrared sensors and laser optic sensors (such as of the types described in U.S. Publication No. US-2016-0267911, which is hereby incorporated herein by reference in its entirety) and may receive and process data or inputs or information from various other sensors and devices (such as a passenger's smart phone or fitness device (wearables), a (non-vehicle inherent) deployable camera or the like), such as of the types and processed in a manner described in the above incorporated U. S. Publication No. US-2017-0274906. Responsive to the various sensor data and inputs for determining vehicle health emergency conditions, the system determines each occupant's tendency to motion sickness as a processing result over long run filtering/profiling monitoring (over multiple vehicle rides by each specific occupant) and the actual, short run filtering/profiling motion sickness assessment of each occupant of the current ride. Thus, the system may store data pertaining to particular individuals and may recognize or identify the particular individuals in future rides so as to accumulate data to assist in determining or assessing motion sickness of the particular individual(s) in the vehicle. Optionally, the occupant having the worst motion sickness assessment may get a higher weight in influencing the vehicle's driving style and way selection than the others.

For preventing or calming down already present motion sickness of one or multiple occupants, the system may generate an alert or message to suggest to these occupants to put away their hand held screens, to stop their movie or game or the system may actively shut down the screens or devices. The threshold or a timeframe of the taking away or shutting down (or allowing) the devices may be part of an optional parent control algorithm, which may be inherent in the vehicle system, collaborating with the connected hand held devices (such as tablets and earphones and smart phones) and vehicle screens or vice versa may be a smart phone app that is (at least partially) controlling the vehicle functions accordingly.

Optionally, for preventing or calming down already present motion sickness of one or multiple occupants, the system may provide these occupants with display of images on monitor screens that show the motion flow of the environmental scene in real time, which may be detected by the outbound or external vehicle sensors, such as vehicle cameras. The scene's screen display may be in high definition (HD) and in a two dimensional (2D) or three dimensional (3D) format or may comprise a light field. The displayed outside scene at the screen may be provided such that the screen's portion, amplification ratio, and virtual viewing angle may always be in a way as been seen from the point of view that the viewing occupant would have when looking at the natural scene by himself or herself (if there would be no blocking of sight, due to the vehicle). In other words, the screen may imitate a (movable) window at the vehicle's naturally non-transparent surfaces. The displayed images thus are displayed on a display screen in the vehicle and are derived from image data captured by an exterior viewing camera of the vehicle, with the exterior viewing camera having a field of view that encompasses the exterior scene that would be viewed by the passenger if the viewed display screen and the portion of the vehicle at which it is mounted were transparent. For example, if the display screen is disposed at a side door of the vehicle, the displayed images may be derived from image data captured by a sideward viewing camera, such that the display screen acts as a virtual window of the vehicle.

There may be areas which may not be captured by any camera, such as the vehicle underground or road surface or the area above the vehicle. Optionally, when the screen's normal axis (directed normally from the screen surface) may be pointed to these areas, the according outside image may be composed artificially. Optionally, real cameras may be attached to or close to the vehicle underbody to capture the underbody scene. Optionally, real cameras may be attached to the vehicle on the top or elsewhere pointing to the top for capturing the outside scene above the vehicle in real time for displaying to an occupant that has been determined to have motion sickness.

The system may utilize aspects of head and face direction and position tracking systems and/or eye tracking systems and/or gesture recognition systems. Such head and face direction and/or position tracking systems and/or eye tracking systems and/or gesture recognition systems may utilize aspects of the systems described in U.S. Publication Nos. US-2016-0137126; US-2015-0352953; US-2015-0296135; US-2015-0294169; US-2015-0232030; US-2015-0022664; US-2015-0015710; US-2015-0009010 and/or US-2014-0336876, which are hereby incorporated herein by reference in their entireties.

The camera or sensor may comprise any suitable camera or sensor. Optionally, the camera may comprise a “smart camera” that includes the imaging sensor array and associated circuitry and image processing circuitry and electrical connectors and the like as part of a camera module, such as by utilizing aspects of the vision systems described in International Publication Nos. WO 2013/081984 and/or WO 2013/081985, which are hereby incorporated herein by reference in their entireties.

The system includes an image processor operable to process image data captured by the camera or cameras, such as for detecting objects or other vehicles or pedestrians or the like in the field of view of one or more of the cameras. For example, the image processor may comprise an image processing chip selected from the EyeQ family of image processing chips available from Mobileye Vision Technologies Ltd. of Jerusalem, Israel, and may include object detection software (such as the types described in U.S. Pat. Nos. 7,855,755; 7,720,580 and/or 7,038,577, which are hereby incorporated herein by reference in their entireties), and may analyze image data to detect vehicles and/or other objects. Responsive to such image processing, and when an object or other vehicle is detected, the system may generate an alert to the driver of the vehicle and/or may generate an overlay at the displayed image to highlight or enhance display of the detected object or vehicle, in order to enhance the driver's awareness of the detected object or vehicle or hazardous condition during a driving maneuver of the equipped vehicle.

The vehicle may include any type of sensor or sensors, such as imaging sensors or radar sensors or lidar sensors or ladar sensors or ultrasonic sensors or the like. The imaging sensor or camera may capture image data for image processing and may comprise any suitable camera or sensing device, such as, for example, a two dimensional array of a plurality of photosensor elements arranged in at least 640 columns and 480 rows (at least a 640×480 imaging array, such as a megapixel imaging array or the like), with a respective lens focusing images onto respective portions of the array. The photosensor array may comprise a plurality of photosensor elements arranged in a photosensor array having rows and columns. Preferably, the imaging array has at least 300,000 photosensor elements or pixels, more preferably at least 500,000 photosensor elements or pixels and more preferably at least 1 million photosensor elements or pixels. The imaging array may capture color image data, such as via spectral filtering at the array, such as via an RGB (red, green and blue) filter or via a red/red complement filter or such as via an RCC (red, clear, clear) filter or the like. The logic and control circuit of the imaging sensor may function in any known manner, and the image processing and algorithmic processing may comprise any suitable means for processing the images and/or image data.

For example, the vision system and/or processing and/or camera and/or circuitry may utilize aspects described in U.S. Pat. Nos. 9,233,641; 9,146,898; 9,174,574; 9,090,234; 9,077,098; 8,818,042; 8,886,401; 9,077,962; 9,068,390; 9,140,789; 9,092,986; 9,205,776; 8,917,169; 8,694,224; 7,005,974; 5,760,962; 5,877,897; 5,796,094; 5,949,331; 6,222,447; 6,302,545; 6,396,397; 6,498,620; 6,523,964; 6,611,202; 6,201,642; 6,690,268; 6,717,610; 6,757,109; 6,802,617; 6,806,452; 6,822,563; 6,891,563; 6,946,978; 7,859,565; 5,550,677; 5,670,935; 6,636,258; 7,145,519; 7,161,616; 7,230,640; 7,248,283; 7,295,229; 7,301,466; 7,592,928; 7,881,496; 7,720,580; 7,038,577; 6,882,287; 5,929,786 and/or 5,786,772, and/or U.S. Publication Nos. US-2014-0340510; US-2014-0313339; US-2014-0347486; US-2014-0320658; US-2014-0336876; US-2014-0307095; US-2014-0327774; US-2014-0327772; US-2014-0320636; US-2014-0293057; US-2014-0309884; US-2014-0226012; US-2014-0293042; US-2014-0218535; US-2014-0218535; US-2014-0247354; US-2014-0247355; US-2014-0247352; US-2014-0232869; US-2014-0211009; US-2014-0160276; US-2014-0168437; US-2014-0168415; US-2014-0160291; US-2014-0152825; US-2014-0139676; US-2014-0138140; US-2014-0104426; US-2014-0098229; US-2014-0085472; US-2014-0067206; US-2014-0049646; US-2014-0052340; US-2014-0025240; US-2014-0028852; US-2014-005907; US-2013-0314503; US-2013-0298866; US-2013-0222593; US-2013-0300869; US-2013-0278769; US-2013-0258077; US-2013-0258077; US-2013-0242099; US-2013-0215271; US-2013-0141578 and/or US-2013-0002873, which are all hereby incorporated herein by reference in their entireties. The system may communicate with other communication systems via any suitable means, such as by utilizing aspects of the systems described in International Publication Nos. WO 2010/144900; WO 2013/043661 and/or WO 2013/081985, and/or U.S. Pat. No. 9,126,525, which are hereby incorporated herein by reference in their entireties.

The system may also communicate with other systems, such as via a vehicle-to-vehicle communication system or a vehicle-to-infrastructure communication system or the like. Such car2car or vehicle to vehicle (V2V) and vehicle-to-infrastructure (car2X or V2X or V2I or 4G or 5G) technology provides for communication between vehicles and/or infrastructure based on information provided by one or more vehicles and/or information provided by a remote server or the like. Such vehicle communication systems may utilize aspects of the systems described in U.S. Pat. Nos. 6,690,268; 6,693,517 and/or 7,580,795, and/or U.S. Publication Nos. US-2014-0375476; US-2014-0218529; US-2013-0222592; US-2012-0218412; US-2012-0062743; US-2015-0251599; US-2015-0158499; US-2015-0124096; US-2015-0352953; US-2016-0036917 and/or US-2016-0210853, which are hereby incorporated herein by reference in their entireties.

The system may utilize sensors, such as radar or lidar sensors or the like. The sensing system may utilize aspects of the systems described in U.S. Pat. Nos. 9,753,121; 9,689,967; 9,599,702; 9,575,160; 9,146,898; 9,036,026; 8,027,029; 8,013,780; 6,825,455; 7,053,357; 7,408,627; 7,405,812; 7,379,163; 7,379,100; 7,375,803; 7,352,454; 7,340,077; 7,321,111; 7,310,431; 7,283,213; 7,212,663; 7,203,356; 7,176,438; 7,157,685; 6,919,549; 6,906,793; 6,876,775; 6,710,770; 6,690,354; 6,678,039; 6,674,895 and/or 6,587,186, and/or International Publication Nos. WO 2018/007995 and/or WO 2011/090484, and/or U.S. Publication Nos. US-2018-0045812; US-2018-0015875; US-2017-0356994; US-2017-0315231; US-2017-0276788; US-2017-0254873; US-2017-0222311 and/or US-2010-0245066 and/or U.S. patent application Ser. No. 15/897,268, filed Feb. 15, 2018 (Attorney Docket MAG04 P-3267R), which are hereby incorporated herein by reference in their entireties.

Changes and modifications in the specifically described embodiments can be carried out without departing from the principles of the invention, which is intended to be limited only by the scope of the appended claims, as interpreted according to the principles of patent law including the doctrine of equivalents.

1. A vehicular control system, said vehicular control system comprising:
a plurality of sensors disposed in a vehicle and having respective fields of sensing that encompass occupants in the vehicle;
a control comprising a processor operable to process data captured by said sensors;
wherein said control, responsive at least in part to processing of data captured by said sensors, determines a likelihood that an individual occupant in the vehicle is developing motion sickness; and
wherein, responsive at least in part to determination that an individual occupant is developing motion sickness, said control performs a function selected from the group consisting of (i) determines an optimized driving route to reduce the causes of motion sickness, (ii) generates a message to the determined individual occupant developing motion sickness, and (iii) generates a display of images for viewing by the determined individual occupant developing motion sickness.

2. The vehicular control system of claim 1, wherein said control determines a likelihood that the individual occupant is developing motion sickness by processing data selected from the group consisting of (i) the individual occupant's body and health data, (ii) data pertaining to the pupils opening of the individual occupant, (iii) data pertaining the skin resistance of the individual occupant, (iv) data pertaining the cold transpiration of the individual occupant, (v) data pertaining the body temperature of the individual occupant, and (vi) data pertaining the heart beat rate of the individual occupant.

3. The vehicular control system of claim 1, wherein the vehicle is autonomously controlled, and wherein said control provides at least one input to the autonomous vehicle control to reduce the likelihood of motion sickness of one or more occupants of the vehicle.

4. The vehicular control system of claim 1, wherein, responsive at least in part to determination that the individual occupant is developing motion sickness, said control determines an optimized driving route to reduce the causes of motion sickness.

5. The vehicular control system of claim 4, wherein said control provides inputs to an autonomous vehicle control that controls the vehicle to follow the determined optimized driving route.

6. The vehicular control system of claim 4, wherein the optimized driving route is determined to provide a route selected from the group consisting of (i) a route having a reduced amount of curves, (ii) a route having a reduced degree of curves, (iii) a route having a reduced number of hills, and (iv) a route having a smoother road surface.

7. The vehicular control system of claim 1, wherein, responsive at least in part to determination that the individual occupant is developing motion sickness, said control generates a message to the determined individual occupant developing motion sickness.

8. The vehicular control system of claim 7, wherein the generated message suggests that the determined individual occupant developing motion sickness stop using a display device.

9. The vehicular control system of claim 8, wherein said control is operable to shut down the display device a threshold period of time following the generated message.

10. The vehicular control system of claim 1, wherein, responsive at least in part to determination that the individual occupant is developing motion sickness, said control generates a display of images for viewing by the determined individual occupant developing motion sickness.

11. The vehicular control system of claim 10, wherein the displayed images provide a display of the exterior scene in real-time.

12. The vehicular control system of claim 11, wherein the displayed images are derived from image data captured by an exterior viewing camera of the vehicle.

13. The vehicular control system of claim 11, wherein the displayed images are displayed on a display screen in the vehicle and the displayed images are derived from image data captured by an exterior viewing camera of the vehicle, with the exterior viewing camera having a field of view that encompasses an exterior scene that would be viewed by the individual occupant if the viewed display screen and the portion of the vehicle at which it is mounted were transparent.

14. A vehicular control system for an autonomous vehicle that is autonomously controlled, said vehicular control system comprising:
a plurality of sensors disposed in an autonomous vehicle and having respective fields of sensing that encompass occupants in the autonomous vehicle;
a control comprising a processor operable to process data captured by said sensors;
wherein said control, responsive at least in part to processing of data captured by said sensors, determines a likelihood that an individual occupant in the autonomous vehicle is developing motion sickness;
wherein said control determines a likelihood that the individual occupant is developing motion sickness by processing data selected from the group consisting of (i) the individual occupant's body and health data, (ii) data pertaining to the pupils opening of the individual occupant, (iii) data pertaining the skin resistance of the individual occupant, (iv) data pertaining the cold transpiration of the individual occupant, (v) data pertaining the body temperature of the individual occupant, and (vi) data pertaining the heart beat rate of the individual occupant; and
wherein, responsive at least in part to determination that an individual occupant is developing motion sickness, said control provides at least one input to the autonomous vehicle control to reduce the likelihood of motion sickness of one or more occupants of the vehicle.

15. The vehicular control system of claim 14, wherein the at least one input comprises an optimized driving route to reduce the causes of motion sickness, and wherein, responsive at least in part to the at least one input, the autonomous vehicle control controls the vehicle to follow the determined optimized driving route.

16. The vehicular control system of claim 15, wherein the optimized driving route is determined to provide a route selected from the group consisting of (i) a route having a reduced amount of curves, (ii) a route having a reduced degree of curves, (iii) a route having a reduced number of hills, and (iv) a route having a smoother road surface.

17. The vehicular control system of claim 14, wherein, responsive at least in part to determination that the individual occupant is developing motion sickness, said control generates a message to the determined individual occupant developing motion sickness.

18. A vehicular control system, said vehicular control system comprising:
a plurality of sensors disposed in a vehicle and having respective fields of sensing that encompass occupants in the vehicle;
a control comprising a processor operable to process data captured by said sensors;
wherein said control, responsive at least in part to processing of data captured by said sensors, determines a likelihood that an individual occupant in the vehicle is developing motion sickness;
wherein said control determines a likelihood that the individual occupant is developing motion sickness by processing data selected from the group consisting of (i) the individual occupant's body and health data, (ii) data pertaining to the pupils opening of the individual occupant, (iii) data pertaining the skin resistance of the individual occupant, (iv) data pertaining the cold transpiration of the individual occupant, (v) data pertaining the body temperature of the individual occupant, and (vi) data pertaining the heart beat rate of the individual occupant; and
wherein, responsive at least in part to determination that an individual occupant is developing motion sickness, said control performs a function selected from the group consisting of (i) generates a message to the determined individual occupant developing motion sickness, and (ii) generates a display of images for viewing by the determined individual occupant developing motion sickness.

19. The vehicular control system of claim 18, wherein the vehicle is autonomously controlled, and wherein said control provides at least one input to the autonomous vehicle control to reduce the likelihood of motion sickness of one or more occupants of the vehicle.

20. The vehicular control system of claim 18, wherein, responsive at least in part to determination that the individual occupant is developing motion sickness, said control generates a display of images for viewing by the determined individual occupant developing motion sickness, and wherein the displayed images provide a display of the exterior scene in real-time, and wherein the displayed images are displayed on a display screen in the vehicle and the displayed images are derived from image data captured by an exterior viewing camera of the vehicle, with the exterior viewing camera having a field of view that encompasses an exterior scene that would be viewed by the individual occupant if the viewed display screen and the portion of the vehicle at which it is mounted were transparent.
